(19) United States
(12) Patent Application Publication
(10) Pub. No.: US 2026/0216160 A1
(43) Pub. Date: Jul. 30, 2026

(54) INJECTABLE COMPOSITIONS OF FLECAINIDE

(71) Applicant: CIPLA LIMITED, Mumbai, Maharashtra (IN)

(72) Inventors: Pradeep BHADAURIA, Weston, FL (US); Arunprasath KALIAPERUMAL, Weston, FL (US); Tushar RAJYAGURU, Bhavnagar, Gujarat (IN); Harshad HARDE, Nashik, Maharashtra (IN); Kamlesh PATKARI, Jalgaon, Maharashtra (IN); Sandip KHAIRNAR, Nashik, Maharashtra (IN)

(21) Appl. No.: 19/145,442

(22) PCT Filed: Jun. 14, 2024
(86) PCT No.: PCT/IN2024/050829

§ 371 Date: Jul. 2, 2025

(30) Foreign Application Priority Data
Jun. 14, 2023 (IN) 202321040602
Jul. 4, 2023 (IN) 202321044923

(51) Int. Cl. A61K 31/4458; A61K 9/00; A61K 47/10; A61K 47/26

(52) U.S. Cl. CPC A61K 31/4458; A61K 9/0019; A61K 47/10; A61K 47/26

(57) ABSTRACT

The present invention discloses injectable pharmaceutical composition comprising flecainide or its pharmaceutically acceptable salts thereof. The invention particularly discloses a ready-to-dilute injectable composition comprising flecainide or its pharmaceutically acceptable salts thereof and at least one tonicity adjusting agent.

### RELATED APPLICATIONS

This application claims priority benefit to Indian Provisional Application No. 202321040602, filed on Jun. 14, 2023, entitled “BUFFER FREE INJECTABLE COMPOSITIONS OF FLECAINIDE”; Indian Provisional Application No. 202321044923, filed on Jul. 4, 2023, entitled “INJECTABLE COMPOSITIONS OF FLECAINIDE”; each of which are incorporated herein by reference for all purposes.

### FIELD OF INVENTION

The present invention relates to injectable pharmaceutical composition comprising flecainide or its pharmaceutically acceptable salts thereof. The invention particularly relates to a ready to dilute injectable composition comprising flecainide or its pharmaceutically acceptable salts thereof and at least one tonicity adjusting agent. The present invention also relates to a ready to use injectable composition comprising flecainide or its pharmaceutically acceptable salts thereof and at least one tonicity adjusting agent. The present invention relates to injectable pharmaceutical composition comprising flecainide or its pharmaceutically acceptable salts thereof wherein said composition is free of sodium. The invention also covers process of preparing the said composition and its use in the treatment of paroxysmal supraventricular tachycardias and paroxysmal atrial fibrillation/flutter.

### BACKGROUND OF THE INVENTION

Atrial fibrillation is an irregular and often very rapid heart rhythm that may cause blood clots in the heart. Atrial fibrillation increases the risk of stroke, heart failure and other heart-related complications. During atrial fibrillation, the atria are irregular and out of rhythm with the ventricles of the heart. This is mostly an asymptomatic phenomenon and may cause fast, pounding heartbeat, palpitations, shortness of breath or weakness. Atrial fibrillations can be intermittent or may be persistent and require proper timely treatment in order to prevent further complications such as stroke.

Treatment for atrial fibrillation may include medications, therapy to reset the heart rhythm and catheter procedures to block erratic heart signals. Atrial flutter is one more complication associated with patients suffering from atrial fibrillation. Although atrial flutter is a different arrhythmia, the treatment is quite similar to atrial fibrillation.

One of the treatment options available for atrial fibrillation and flutter is Flecainide. Flecainide is approved in the United States as an oral dosage form in the form an immediate release tablet under the brand name Tambocor®. It can be seen from the label of Tambocor® that Flecainide is administered under medical supervision and the dose is decided depending on the cardiac parameters of the patients. Thus, there is a need to accurately and precisely administer a dose which is suitable to a subject in need thereof. The inventors of the present invention formulated an injectable dosage form of flecainide which provides the flexibility of administering a dose in an accurate, precise and convenient manner as compared to administering a solid dosage form. Flecainide is approved as an injectable dosage form in European countries. However, it was never approved in the United States. Further, the injectable dosage form approved in Europe is a ready to dilute injection which contains sodium acetate as buffer. It is well known that sodium ions can increase the chances of developing atrial fibrillation or can worsen existing atrial fibrillation condition. This is due to tendency of sodium ions to elevate the blood pressure. Elevated blood pressure can increase the risk of developing atrial fibrillation. Thus, it would be detrimental to administer an injection composition containing sodium to a patient suffering from atrial fibrillation. The invention addresses this unmet need and relates to an injectable composition of flecainide which is free of sodium.

Further, the injectable dosage form approved in Europe is a ready to dilute injection which needs to be diluted with compatible admixture diluents and the package insert mentions that such mixture upon dilution is not stable upon dilution for more than 24 hours. Thus, there is a need for an injectable composition of flecainide or its pharmaceutically acceptable salts thereof which remains stable throughout the shelf life. The present invention addresses this unmet need and relates to a ready to use injectable composition of flecainide which is stable throughout the shelf life. Further, such a composition is free of sodium, which can be safely administered to patients suffering from atrial flutter and atrial fibrillation.

The inventors of the present invention through rigorous experimentation have designed an injectable composition of flecainide or its pharmaceutically acceptable salts thereof which remains stable throughout the shelf life. Further, the inventors through their experimentation have formulated a ready to use injectable composition of flecainide or its pharmaceutically acceptable salts thereof, which is stable over the entire shelf life of the product and does not require dilution with compatible admixture diluents before administration to a subject in need thereof.

### OBJECT OF THE INVENTION

An object of the invention is to an injectable pharmaceutical composition comprising flecainide or its pharmaceutically acceptable salts thereof and at least one tonicity adjusting agent.

Another object of the invention relates to a ready to dilute composition comprising flecainide or its pharmaceutically acceptable salts thereof and at least one tonicity adjusting agent.

A further object of the invention also relates to a ready to use composition comprising flecainide or its pharmaceutically acceptable salts thereof and at least one tonicity adjusting agent.

One more object of the invention relates to an injectable pharmaceutical composition comprising flecainide or its pharmaceutically acceptable salts thereof, wherein said composition is free of sodium.

### SUMMARY OF THE INVENTION

The present invention relates to injectable pharmaceutical composition comprising flecainide or its pharmaceutically acceptable salts thereof. The invention particularly relates to a ready to dilute injectable composition comprising flecainide or its pharmaceutically acceptable salts thereof and at least one tonicity adjusting agent. The present invention also relates to a ready to use injectable composition comprising flecainide or its pharmaceutically acceptable salts thereof and at least one tonicity adjusting agent. The present invention relates to injectable pharmaceutical composition comprising flecainide or its pharmaceutically acceptable salts thereof wherein said composition is free of sodium. The invention also covers process of preparing the said composition and its use in the treatment of paroxysmal supraventricular tachycardias and paroxysmal atrial fibrillation/flutter.

The present invention relates to an injectable composition comprising flecainide or its pharmaceutically acceptable salts thereof and at least one tonicity adjusting agent.

In one embodiment, the injectable composition of the present invention is a ready to use composition.

In another embodiment, the injectable composition is a ready to dilute composition.

In a further embodiment, the injectable composition comprises about 50 to about 500 mg flecainide or its pharmaceutically acceptable salts thereof.

In an embodiment, the injectable composition comprises about 150 mg flecainide or its pharmaceutically acceptable salts thereof.

In one more embodiment, the injectable composition comprises at least one tonicity adjusting agent. In one further embodiment, the at least one tonicity adjusting agent selected from dextrose, lactose, sorbitol, sucrose, mannitol, trehalose, raffinose, polyethylene glycol, hydroxyethyl starch, glycine, propylene glycol and ethanol.

In another embodiment, the injectable composition comprises about 1 mg to about 30000 mg of at least one tonicity adjusting agent. In a preferred embodiment, the injectable composition comprises about 100 mg to about 20000 mg of at least one tonicity adjusting agent.

In an embodiment, the injectable composition comprises about 10 mg/ml to about 150 mg/ml of at least one tonicity adjusting agent. In one embodiment, the injectable composition comprises about 150 mg flecainide or its pharmaceutically acceptable salts thereof, about 10 mg/ml to about 150 mg/ml propylene glycol and water for injection. In one more embodiment, the injectable composition comprises about 150 mg flecainide or its pharmaceutically acceptable salts thereof, about 20 mg/ml propylene glycol and water for injection.

In one embodiment, the injectable composition is a ready to dilute composition and comprises about 100 mg to about 1000 mg of at least one tonicity adjusting agent. In another embodiment, the ready to dilute composition comprises about 180 mg ethanol. In one more embodiment, the ready to dilute composition comprises about 300 mg propylene glycol. In a further embodiment, the ready to dilute composition comprises about 750 mg dextrose. In one embodiment, the ready to dilute composition comprises about 182 mg ethanol. In one embodiment, the ready to dilute composition has a total volume of about 15 ml.

In one more embodiment, the injectable composition is a ready to use composition and comprises about 2000 mg to about 20000 mg of at least one tonicity adjusting agent. In another embodiment, the ready to dilute composition comprises about 3000 mg propylene glycol. In a further embodiment, the ready to dilute composition comprises about 18000 mg polyethylene glycol. In one more embodiment, the ready to dilute composition comprises about 7500 mg dextrose. In one embodiment, the ready to use composition has a total volume of about 150 ml.

In one more embodiment, the injectable composition comprises a pharmaceutically acceptable excipient comprising at least one pharmaceutically acceptable vehicle. Suitable examples of pharmaceutically acceptable vehicle include, but are not limited to water for injection, and the like.

In an embodiment, the injectable composition has a fill volume of about 1 ml to about 250 ml.

In another embodiment, the injectable composition has a fill volume of about 1 ml to about 15 ml.

In a further embodiment, the injectable composition has a fill volume of about 50 ml to about 150 ml.

In a preferred embodiment, the injectable composition has a fill volume of about 15 ml. In another preferred embodiment, the injectable composition has a fill volume of about 150 ml.

In another embodiment, the injectable composition has a pH of about 5 to about 9, more preferably about 6 to about 8.

In one other embodiment, the injectable composition has osmolality of about 200 mOsm to about 500 mOsm.

In one preferred embodiment, the injectable composition comprises about 150 mg flecainide or its pharmaceutically acceptable salts thereof, about 600 mg to about 900 mg dextrose and water for injection.

In a preferred embodiment, the injectable composition comprises about 150 mg flecainide or its pharmaceutically acceptable salts thereof, about 150 mg to about 250 mg ethanol and water for injection.

In another preferred embodiment, the injectable composition comprises about 150 mg flecainide or its pharmaceutically acceptable salts thereof, about 6000 mg to about 9000 mg dextrose and water for injection.

In one preferred embodiment, the injectable composition comprises about 150 mg flecainide or its pharmaceutically acceptable salts thereof, about 150 mg to about 4000 mg propylene glycol and water for injection.

In one more preferred embodiment, the injectable composition comprises about 150 mg flecainide or its pharmaceutically acceptable salts thereof, about 15000 mg to about 20000 mg polyethylene glycol and water for injection.

In one preferred embodiment, the injectable composition comprises about 150 mg flecainide or its pharmaceutically acceptable salts thereof, about 750 mg dextrose and water for injection.

In a preferred embodiment, the injectable composition comprises about 150 mg flecainide or its pharmaceutically acceptable salts thereof, about 182 mg ethanol and water for injection.

In another preferred embodiment, the injectable composition comprises about 150 mg flecainide or its pharmaceutically acceptable salts thereof, about 180 mg ethanol and water for injection.

In one preferred embodiment, the injectable composition comprises about 150 mg flecainide or its pharmaceutically acceptable salts thereof, about 300 mg propylene glycol and water for injection.

In another preferred embodiment, the injectable composition comprises about 150 mg flecainide or its pharmaceutically acceptable salts thereof, about 7500 mg dextrose and water for injection.

In one preferred embodiment, the injectable composition comprises about 150 mg flecainide or its pharmaceutically acceptable salts thereof, about 3000 mg propylene glycol and water for injection.

In one preferred embodiment, the injectable composition comprises about 150 mg flecainide or its pharmaceutically acceptable salts thereof, about 18000 mg polyethylene glycol and water for injection.

In one embodiment, the injectable composition comprising flecainide or its pharmaceutically acceptable salts thereof, further contains a buffer.

In a further embodiment, the injectable composition comprising flecainide or its pharmaceutically acceptable salts thereof is free of buffer.

In one more embodiment, the buffer free injectable composition of the present invention is a ready to use composition.

In another embodiment, the buffer free injectable composition is a ready to dilute composition.

In an embodiment, the injectable composition comprising flecainide or its pharmaceutically acceptable salts thereof is free of sodium.

In one another embodiment, the injectable composition comprising flecainide or its pharmaceutically acceptable salts thereof remains stable at room temperature for a period of about 3 months.

In one embodiment, the injectable composition comprising flecainide or its pharmaceutically acceptable salts thereof remains stable at room temperature for a period of about 6 months.

In another embodiment, the injectable composition comprising flecainide or its pharmaceutically acceptable salts thereof remains stable at room temperature for a period of about 1 year.

In one more embodiment, the injectable composition comprising flecainide or its pharmaceutically acceptable salts thereof remains stable at room temperature for a period of about 2 years.

In one embodiment, the injectable composition comprising flecainide or its pharmaceutically acceptable salts thereof remains stable at room temperature for a period of about 3 years.

### DETAILED DESCRIPTION

The present invention relates to injectable pharmaceutical composition comprising flecainide or its pharmaceutically acceptable salts thereof. The invention particularly relates to a ready to dilute injectable composition comprising flecainide or its pharmaceutically acceptable salts thereof and at least one tonicity adjusting agent. The present invention also relates to a ready to use injectable composition comprising flecainide or its pharmaceutically acceptable salts thereof and at least one tonicity adjusting agent. The present invention relates to injectable pharmaceutical composition comprising flecainide or its pharmaceutically acceptable salts thereof wherein said composition is free of sodium. The invention also covers process of preparing the said composition and its use in the treatment of paroxysmal supraventricular tachycardias and paroxysmal atrial fibrillation/flutter.

Flecainide or its pharmaceutically acceptable salts thereof is designated chemically as N-(piperidin-2-ylmethyl)-2,5-bis(2,2,2-trifluoroethoxy)benzamide. Flecainide has a molecular formula C17H20F6N2O3 and has the following structure:

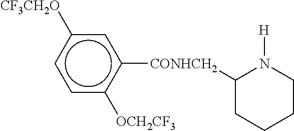

Flecainide is approved in the United States as an oral dosage form in the form an immediate release tablet under the brand name Tambocor®. It can be seen from the label of Tambocor® that Flecainide is administered under medical supervision and the dose is decided depending on the cardiac parameters of the patients. Thus, there is a need to accurately and precisely administer a dose which is suitable to a subject in need thereof. The inventors of the present invention formulated an injectable dosage form of flecainide which provides the flexibility of administering a dose in an accurate, precise and convenient manner as compared to administering a solid dosage form. Flecainide is approved as an injectable dosage form in European countries. However, it was never approved in the United States. Further, the injectable dosage form approved in Europe is a ready to dilute injection which contains sodium acetate as buffer. It is well known that sodium ions can increase the chances of developing atrial fibrillation or can worsen existing atrial fibrillation condition. This is due to tendency of sodium ions to elevate the blood pressure. Elevated blood pressure can increase the risk of developing atrial fibrillation. Thus, it would be detrimental to administer an injection composition containing sodium to a patient suffering from atrial fibrillation. The invention addresses this unmet need and relates to an injectable composition of flecainide which is free of sodium. Further, the injectable dosage form approved in Europe is a ready to dilute injection which needs to be diluted with compatible admixture diluents and the package insert mentions that such mixture upon dilution is not stable upon dilution for more than 24 hours. Thus, there is a need for an injectable composition of flecainide or its pharmaceutically acceptable salts thereof which remains stable throughout the shelf life. The present invention addresses this unmet need and relates to a ready to use injectable composition of flecainide which is stable throughout the shelf life. Further, such a composition is free of sodium, which can be safely administered to patients suffering from atrial flutter and atrial fibrillation.

The inventors of the present invention through rigorous experimentation have designed an injectable composition of flecainide or its pharmaceutically acceptable salts thereof which remains stable throughout the shelf life. Further, the inventors through their experimentation have formulated a ready to use injectable composition of flecainide or its pharmaceutically acceptable salts thereof, which is stable over the entire shelf life of the product and does not require dilution with compatible admixture diluents before administration to a subject in need thereof.

The term “flecainide” as used herein includes flecainide or its pharmaceutically acceptable salts which are well known in the art and also embrace any polymorph, pseudo-polymorph, solvate, hydrate, crystalline or amorphous form of flecainide and any prodrug of flecainide which can be delivered in a vehicle and manner described herein for flecainide. The salts can be prepared in situ during the final isolation and purification of the compounds of the invention, or separately by reacting the pharmaceutically active substance, having a freebase function, with a suitable organic acid or inorganic acid.

The injectable composition of the present invention may comprise about 50 mg to about 500 mg flecainide or its pharmaceutically acceptable salts thereof. Preferably, the injectable composition of the present invention may comprise about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, or about 500 mg flecainide or its pharmaceutically acceptable salts thereof. In some embodiments, the injectable composition contains about 50 mg to about 200 mg flecainide or its pharmaceutically acceptable salt thereof, more preferably about 100 mg to about 200 mg. In a specific embodiment, the injectable composition contains about 150 mg flecainide or a pharmaceutically acceptable salt thereof.

The following description sets forth exemplary embodiments of the present technology. It should be recognized, however, that such description is not intended as a limitation on the scope of the present disclosure but is instead provided as a description of exemplary embodiments.

As used in the present specification, the following words, phrases and symbols are generally intended to have the meanings as set forth below, except to the extent that the context in which they are used indicates otherwise.

Reference to “about” a value or parameter herein includes (and describes) embodiments that are directed to that value or parameter per se. In certain embodiments, the term “about” includes the indicated amount±10%. In other embodiments, the term “about” includes the indicated amount±5%. In certain other embodiments, the term “about” includes the indicated amount±1%. Also, to the term “about X” includes description of “X”. Also, the singular forms “a” and “the” include plural references unless the context clearly dictates otherwise. Thus, e.g., reference to “the compound” includes a plurality of such compounds and reference to “the assay” includes reference to one or more assays and equivalents thereof known to those skilled in the art.

The injectable composition of the present invention may further comprise at least one tonicity adjusting agent. Suitable examples of tonicity adjusting agent include, but are not limited to dextrose, lactose, sorbitol, sucrose, mannitol, trehalose, raffinose, polyethylene glycol, hydroxyethyl starch, glycine, propylene glycol and ethanol.

In another embodiment, the injectable composition comprises about 1 mg to about 30000 mg of at least one tonicity adjusting agent. In a preferred embodiment, the injectable composition comprises about 100 mg to about 20000 mg of at least one tonicity adjusting agent. In an embodiment, the injectable composition comprises about 10 mg/ml to about 150 mg/ml of at least one tonicity adjusting agent.

In one embodiment, the injectable composition comprises about 150 mg flecainide or its pharmaceutically acceptable salts thereof, about 600 mg to about 900 mg dextrose and water for injection.

In an embodiment, the injectable composition comprises about 150 mg flecainide or its pharmaceutically acceptable salts thereof, about 150 mg to about 250 mg ethanol and water for injection.

In another embodiment, the injectable composition comprises about 150 mg flecainide or its pharmaceutically acceptable salts thereof, about 6000 mg to about 9000 mg dextrose and water for injection.

In one another embodiment, the injectable composition comprises about 150 mg flecainide or its pharmaceutically acceptable salts thereof, about 150 mg to about 4000 mg propylene glycol and water for injection.

In one more embodiment, the injectable composition comprises about 150 mg flecainide or its pharmaceutically acceptable salts thereof, about 15000 mg to about 20000 mg polyethylene glycol and water for injection.

In one embodiment, the injectable composition is a ready to dilute composition and comprises about 100 mg to about 1000 mg of at least one tonicity adjusting agent.

In another embodiment, the ready to dilute composition comprises about 180 mg ethanol. In one more embodiment, the ready to dilute composition comprises about 300 mg propylene glycol. In a further embodiment, the ready to dilute composition comprises about 750 mg dextrose. In one embodiment, the ready to dilute composition comprises about 182 mg ethanol. In one embodiment, the ready to dilute composition has a total volume of about 15 ml.

In one more embodiment, the injectable composition is a ready to use composition and comprises about 2000 mg to about 20000 mg of at least one tonicity adjusting agent. In another embodiment, the ready to use composition comprises about 3000 mg propylene glycol. In a further embodiment, the ready to use composition comprises about 18000 mg polyethylene glycol. In one more embodiment, the ready to use composition comprises about 7500 mg dextrose. In one embodiment, the ready to use composition has a total volume of about 150 ml.

In another embodiment, the injectable composition comprises about 150 mg flecainide or its pharmaceutically acceptable salts thereof, about 10 mg/ml to about 150 mg/ml of at least one tonicity adjusting agent and water for injection.

In one embodiment, the injectable composition comprises about 150 mg flecainide or its pharmaceutically acceptable salts thereof, about 10 mg/ml to about 150 mg/ml propylene glycol and water for injection.

In one more embodiment, the injectable composition comprises about 150 mg flecainide or its pharmaceutically acceptable salts thereof, about 20 mg/ml propylene glycol and water for injection.

In an embodiment, the injectable composition comprises about 150 mg flecainide or its pharmaceutically acceptable salts thereof, about 10 mg/ml to about 150 mg/ml dextrose and water for injection.

In one embodiment, the injectable composition comprises about 150 mg flecainide or its pharmaceutically acceptable salts thereof, about 10 mg/ml to about 150 mg/ml ethanol and water for injection.

In a further embodiment, the injectable composition comprises about 150 mg flecainide or its pharmaceutically acceptable salts thereof, about 10 mg/ml to about 150 mg/ml polyethylene glycol and water for injection.

The injectable composition of the present invention may comprise a buffering agent. Buffers, include, but are not limited to ascorbate, lactobionate, gentisate, succinate, α-lipoic acid, maleate, chloroacetate, bicarbonate, tartrate, glycylglycine, formate, benzoate, citrate, lactate, acetate, phosphate, propionate, pyridine, piperazine, pyrophosphate, histidine, 2-(N-morpholine)ethanesulfonic acid (“MES”), cacodylic acid, (bis(2-hydroxyethyl)-imino-tris(hydroxymethyl)-methane) (“bis-TRIS”), bicarbonate, amino acids (glycine, glutamate, aspartate and etc) or a combination of these buffering agents.

The injectable composition of the present invention may further comprise at least one pharmaceutically acceptable vehicle. Suitable examples of pharmaceutically acceptable vehicle include, but are not limited to water for injection and the like.

The injectable composition of the present invention may have a fill volume comprising from about 1 ml to about 250 ml. Preferably, the injectable composition of the present invention may have a fill volume comprising about 15 ml. Alternatively, a preferred embodiment may comprise an injectable composition having a fill volume of about 150 ml.

The pH of the injectable composition of the present invention may range from about 5 to about 9, more preferably about 6 to about 8, including all subranges therebetween. Preferably, the injectable composition of the present invention may have a pH of about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, or about 8. The pH may be adjusted with the help of pH adjusting agents well known in the art. In one embodiment, the injectable composition may not contain any pH adjusting agents. The injectable composition of the present invention may be hypotonic, isotonic or hypertonic, preferably isotonic. In one embodiment, the injectable composition of the present invention has an osmolality of about 200 mOsm to about 500 mOsm.

The present invention relates an injectable composition, wherein such a composition is a ready to dilute composition. In an embodiment, the ready to dilute composition comprises about 150 mg flecainide, about 100 mg to about 1000 mg of at least one tonicity adjusting agent and water for injection. In one embodiment, the ready to dilute injectable composition comprises about 150 mg flecainide, about 750 mg dextrose and water for injection. In another embodiment, the ready to dilute injectable composition comprises about 150 mg flecainide, about 182 mg ethanol and water for injection. In one more embodiment, the ready to dilute injectable composition comprises about 150 mg flecainide, about 180 mg ethanol and water for injection. In a further embodiment, the ready to dilute injectable composition comprises about 150 mg flecainide or its pharmaceutically acceptable salts thereof, about 300 mg propylene glycol and water for injection. In one embodiment, the ready to dilute composition has a total volume of about 15 ml.

The present invention relates an injectable composition, wherein such a composition is a ready to use composition. In an embodiment, the ready to use composition comprises about 150 mg flecainide, about 2000 mg to about 20000 mg of at least one tonicity adjusting agent and water for injection. In one embodiment, the ready to use injectable composition comprises about 150 mg flecainide, about 7500 mg dextrose and water for injection. In one more embodiment, the ready to use injectable composition comprises about 150 mg flecainide or its pharmaceutically acceptable salts thereof, about 3000 mg propylene glycol and water for injection. In one another embodiment, the ready to use injectable composition comprises about 150 mg flecainide or its pharmaceutically acceptable salts thereof, about 18000 mg polyethylene glycol and water for injection. In one embodiment, the ready to use composition has a total volume of about 150 ml.

In one embodiment, the injectable composition of the present invention is stable throughout the shelf life. The term “stable” refers to an injectable composition of the present invention which is physically as well as chemically stable as demonstrated by compliance to acceptable specification when the composition is stored at convenient temperature, such as between about 0° C. and about 60° C., for a commercially reasonable period of time, such as at least about 1 day, at least about 1 week, at least about 1 month, at least about 3 months, at least about 6 months, at least about 1 year, at least about 2 years, or at least about 3 years. The term “stable” also refers to an injectable composition as per the present invention which exhibits physical stability for a sufficiently long time to allow for infusion of the product upon dilution with compatible admixture diluent such as sterile water for injection, saline or dextrose. The injectable compositions of the present invention remain stable after dilution with a compatible admixture diluent for a sufficient time to allow for infusion to be completed. Suitably, the injectable composition of flecainide of the present invention remains physically stable, with no precipitation or crystallization or color change upon storage and the shelf-life period of 18-24 months. Suitably, injectable composition of flecainide remains chemically stable, wherein various parameters such as the drug content (assay of flecainide) and content of related substances, i.e. known and unknown impurities remains within specified limits such as those specified according to ICH guidelines, upon storage for prolonged period of time such as for at least about 3 months, at least about 6 months, at least about 1 year, at least about 2 years, at least about 3 years or longer. The injectable composition of present invention is substantially free of impurities. For purposes of the present invention, “substantially free of impurities’ shall be understood to include flecainide containing injectable compositions in which the amount of total impurities is less than about 5% of the sum of peak areas of all degradants, as calculated on a normalized peak area response (“PAR) basis as determined by high performance liquid chromatograph (“HPLC) after a period of about 18 months at a temperature of from about 2° C. to about 8° C. The amount of impurities is further calculated as being based upon the original amount of flecainide being present in the composition. Preferably, the said injectable compositions of flecainide prevent degradation of flecainide such that not more than 2%, not more than 1%, not more than 0.4%, not more than 0.2% of flecainide impurity or impurities are formed over the storage period. In yet another preferred embodiment the value of assay of flecainide remains within the specified limit of 90-110% by weight of the label claim.

The ICH storage stability studies were performed on the injectable composition of the present invention packaged in the proposed commercial primary packaging and closure system. The stability study samples were stored at 2-8° C., 25° C., 30° C., and 40° C. The necessary parameters viz., assay, related substances, particle size distribution, pH, and osmolality were tested and found to be within specification at both stability conditions. In some preferred aspects of the invention, the time for which long term storage are contemplated include periods of at least about 24 months or longer with such that the composition is substantially free of impurities. Preferably, the process of preparation of the injectable composition of the present invention is carried out under nitrogen purging or blanketing. The injectable composition of the present invention may be sterilized under aseptic conditions well known in the art, preferably sterilization by filtration.

The injectable compositions of the present invention can be packaged in any suitable sterile vial or prefilled syringe or a bag or any container fit for the sterile storage of the composition. The injectable composition of the present invention can be provided in a kit or package that includes a container enclosing the composition. Suitable containers can be glass vials, i.e. Schott treated vials, molded glass vials, and CZ resin vials, polypropylene or polyethylene vials or other special purpose containers. Suitable containers can be prefilled syringe such as glass prefilled syringes, plastic prefilled syringes. Containers are of a size sufficient to hold one or more doses of flecainide. The container may be part of a syringe or separate from the syringe. The kit or package also includes a needle that can be suitably mounted to the syringe. The size of the needle, in some embodiments, is equal to or smaller than 18 G, 19 G, 20 G, 21 G, 22 G, 23 G, 24 G, or 25 G. In one embodiment, the needle has a size that is 20 G or smaller. In one embodiment, the needle has a size that is 21 G or smaller. In one embodiment, the needle has a size that is 22 G or smaller. In one embodiment, the needle has a size that is 23 G or smaller. In one embodiment, the injectable composition of the present invention may be administered without the need of an in-line filter during administration, demonstrating the absence of particulate matter throughout its shelf life. In another embodiment, the injectable composition of the present invention may be filtered by using an in-line filter before administration.

The injectable composition of the present invention may be a ready to dilute composition. In one embodiment, such composition may comprise about 150 mg flecainide and at least one tonicity adjusting agent, and wherein the fill volume of composition is about 15 ml. The ready to dilute composition can be diluted in appropriate compatible admixture diluents such as water for injection before administration to a subject in need thereof.

The injectable composition of the present invention may be a ready to use composition. In one embodiment, such composition may comprise about 150 mg flecainide and at least one tonicity adjusting agent, and wherein the fill volume of composition is about 150 ml. In an embodiment, such composition can be directly administered to a patient in need thereof without further dilution in any appropriate compatible admixture diluents.

### EXAMPLES

The following examples are for the purpose of illustration of the invention only and are not intended to limit the scope of the present invention in any manner whatsoever.

### Example 1

The below example is a ready to dilute injectable composition comprising flecainide.

| Ingredients | Quantity in mg |
|---|---|
| Flecainide | 150 |
| Dextrose | 750 |
| Water for injection | q.s. 15 ml |
| pH | 7.24 |
| Osmolality (mOsmol/kg) | 342 |

### Manufacturing Process:

1. Dextrose was added in water for injection
2. Flecainide was added to the solution of step 1 till a clear solution was obtained.
3. Volume of solution of step 2 was made up with water for injection.

The below table demonstrates the stability of the composition of Example 1.

| | | 25° C./60% RH | | 40° C./75% RH |
|---|---|---|---|---|
| Parameters | Initial | 3 M | 6 M | 6 M |
| Description | Clear | Clear | Clear | Clear |
| | Colourless | Colourless | Colourless | Colourless |
| | Solution | Solution | Solution | Solution |
| pH | 7.24 | 7.2 | 7.0 | 6.55 |
| Assay (%) | 100.50 | 101.7 | 100.6 | 99.8 |
| Total | <0.1 | <0.1 | <0.1 | <0.1 |
| Impurities | | | | |
| (%) | | | | |

### Example 2

The below example is a ready to dilute injectable composition comprising flecainide.

| Ingredients | Quantity in mg |
|---|---|
| Flecainide | 150 |
| Ethanol | 182 |
| Water for injection | q.s. 15 ml |
| pH | 7.18 |
| Osmolality (mOsmol/kg) | 323 |

### Manufacturing Process:

1. Ethanol was added in water for injection
2. Flecainide was added to the solution of step 1 till a clear solution was obtained.
3. Volume of solution of step 2 was made up with water for injection.

### Example 3

The below example is a ready to dilute injectable composition comprising flecainide.

| Ingredients | Quantity in mg |
|---|---|
| Flecainide | 150 |
| Ethanol | 180 |
| Water for injection | q.s. 15 ml |
| pH | 7.2 |
| Osmolality (mOsmol/kg) | 323 |

### Manufacturing Process:

1. Ethanol was added in water for injection
2. Flecainide was added to the solution of step 1 till a clear solution was obtained.
3. Volume of solution of step 2 was made up with water for injection.

### Example 4

The below example is a ready to dilute injectable composition comprising flecainide.

| Ingredients | Quantity in mg |
|---|---|
| Flecainide | 150 |
| Propylene glycol | 300 |
| Water for injection | q.s. 15 ml |
| pH | 7.02 |
| Osmolality (mOsmol/kg) | 325 |

### Manufacturing Process:

1. Propylene glycol was added in water for injection
2. Flecainide was added to the solution of step 1 till a clear solution was obtained.
3. Volume of solution of step 2 was made up with water for injection.

The below table demonstrates the stability of the composition of Example 4.

| | | 25° C./60% RH | | 30° C./75% RH | | 40° C./75% RH | |
|---|---|---|---|---|---|---|---|
| Parameters | Initial | 1 M | 3 M | 1 M | 3 M | 1 M | 3 M |
| Description | Clear | Clear | Clear | Clear | Clear | Clear | Clear |
| | Colour- | Colour- | Colour- | Colour- | Colour- | Colour- | Colour- |
| | less | less | less | less | less | less | less |
| | Solution | Solution | Solution | Solution | Solution | Solution | Solution |
| pH | 6.95 | 7.17 | 7.15 | 7.15 | 7.16 | 7.12 | 7.21 |
| Assay (%) | 102.2 | 101.4 | 102.2 | 101.6 | 103.2 | 101.4 | 101.7 |
| Total | 0.14 | 0.14 | 0.25 | 0.13 | 0.26 | 0.23 | 0.28 |
| Impurities | | | | | | | |
| (%) | | | | | | | |

### Example 5

The below example is a ready to use injectable composition comprising flecainide.

| Ingredients | Quantity in mg |
|---|---|
| Flecainide | 150 |
| Propylene glycol | 3000 |
| Water for injection | q.s. 150 ml |
| pH | 6.76 |
| Osmolality | 267 |

### Manufacturing Process:

1. Propylene glycol was added in water for injection
2. Flecainide was added to the solution of step 1 till a clear solution was obtained.
3. Volume of solution of step 2 was made up with water for injection.

The below table demonstrates the stability of the composition of Example 5.

| | | 25° C./60% RH | | 35° C./75% RH | 40° C./75% RH | |
|---|---|---|---|---|---|---|
| Parameters | Initial | 1 M | 3 M | 3 M | 1 M | 3 M |
| Description | Clear | Clear | Clear | Clear | Clear | Clear |
| | Colour- | Colour- | Colour- | Colour- | Colour- | Colour- |
| | less | less | less | less | less | less |
| | Solution | Solution | Solution | Solution | Solution | Solution |
| pH | 6.66 | 6.47 | 6.71 | 6.83 | 6.52 | 6.5 |
| Assay (%) | 99.4 | 98.8 | 101.7 | 101.6 | 98.7 | 101.3 |
| Total | <0.1 | <0.1 | <0.1 | <0.1 | <0.1 | <0.1 |
| Impurities | | | | | | |
| (%) | | | | | | |

### Example 6

The below example is a ready to use injectable composition comprising flecainide.

| Ingredients | Quantity in mg |
|---|---|
| Flecainide | 150 |
| Polyethylene glycol | 18000 |
| Water for injection | q.s. 150 ml |
| pH | 7.35 |
| Osmolality | 285 |

### Manufacturing Process:

1. Polyethylene glycol was added in water for injection
2. Flecainide was added to the solution of step 1 till a clear solution was obtained.
3. Volume of solution of step 2 was made up with water for injection.

### Example 7

The below example is a ready to use injectable composition comprising flecainide.

| Ingredients | Quantity in mg |
|---|---|
| Flecainide | 150 |
| Dextrose | 7500 |
| Water for injection | q.s. 150 ml |
| pH | 7.01 |
| Osmolality (mOsmol/kg) | 306 |

### Manufacturing Process:

1. Dextrose was added in water for injection
2. Flecainide was added to the solution of step 1 till a clear solution was obtained.
3. Volume of solution of step 2 was made up with water for injection.

While the invention has been described in connection with specific embodiments thereof. it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth.

1-24. (canceled)

25. An injectable composition comprising flecainide, or a pharmaceutically acceptable salt thereof, and at least one tonicity adjusting agent.

26. The injectable composition of claim 25, further comprising water.

27. The injectable composition of claim 25, wherein said composition is a ready-to-use composition.

28. The injectable composition of claim 25, wherein said composition comprises about 50 mg to about 500 mg flecainide, or its pharmaceutically acceptable salts thereof.

29. The injectable composition of claim 25, wherein said composition comprises about 100 mg to about 200 mg flecainide, or a pharmaceutically acceptable salt thereof.

30. The injectable composition of claim 25, wherein said composition comprises about 50 mg flecainide, about 75 mg flecainide, about 100 mg flecainide, about 125 mg flecainide, about 150 mg flecainide, about 175 mg flecainide, about 200 mg flecainide, about 225 mg flecainide, about 250 mg flecainide, about 275 mg flecainide, about 300 mg flecainide, about 325 mg flecainide, about 350 mg flecainide, about 375 mg flecainide, about 400 mg flecainide, about 425 mg flecainide, about 450 mg flecainide, about 475 mg flecainide, about 500 mg flecainide, or a pharmaceutically acceptable salt thereof.

31. The injectable composition of claim 25, wherein said composition comprises about 150 mg flecainide, or its pharmaceutically acceptable salts thereof.

32. The injectable composition of claim 25, wherein said at least one tonicity adjusting agent is selected from dextrose, lactose, calcium chloride, magnesium chloride, sorbitol, sucrose, mannitol, trehalose, raffinose, polyethylene glycol, hydroxyethyl starch, glycine, propylene glycol, and ethanol.

33. The injectable composition of claim 25, wherein said at least one tonicity adjusting agent is selected from dextrose, propylene glycol, sucrose, mannitol, polyethylene glycol, and ethanol.

34. The injectable composition of claim 25, wherein said composition comprises about 1 mg to about 30,000 mg of at least one tonicity adjusting agent.

35. The injectable composition of claim 25, wherein said composition comprises about 100 mg to about 20,000 mg of at least one tonicity adjusting agent.

36. The injectable composition of claim 25, wherein said composition comprises about 10 mg/ml to about 150 mg/ml of at least one tonicity adjusting agent.

37. The injectable composition of claim 25, wherein said composition comprises about 10 mg/ml to about 150 mg/ml of dextrose.

38. The injectable composition of claim 25, wherein said composition comprises about 50 mg to about 500 mg flecainide or its pharmaceutically acceptable salts thereof and about 10 mg/ml to about 150 mg/ml tonicity adjusting agent.

39. The injectable composition of claim 25, wherein said composition comprises about 150 mg flecainide or its pharmaceutically acceptable salts thereof and about 10 mg/ml to about 150 mg/ml tonicity adjusting agent.

40. The injectable composition of claim 25, wherein said composition further comprises a buffer.

41. The injectable composition of claim 25, wherein said composition is free of buffer.

42. The injectable composition of claim 25, wherein said composition is free of sodium.

43. The injectable composition of claim 25, wherein said composition has a fill volume of about 1 ml to about 250 ml.

44. The injectable composition of claim 25, wherein said composition has a fill volume of about 50 ml to about 150 ml.

45. The injectable composition of claim 25, wherein said composition has a pH of about 6 to about 8.

46. The injectable composition of claim 25, having a pH of 6.76-7.24.

47. The injectable composition of claim 25, wherein said composition has osmolality of about 200 mOsm to about 500 mOsm.

48. The injectable composition of claim 25, having an osmolality of 267-325 mOsmol/kg.

49. The injectable composition of claim 25, that has been sterilized under aseptic conditions.

50. The injectable composition of claim 25, that has been sterilized by filtration.

51. The injectable composition of claim 25, that has been sterilized by aseptic filtration.

52. The injectable composition of claim 25, having a purity, such that the total impurities is less than 0.28%.

53. The injectable composition of claim 25, having a purity, such that the total impurities is less than 0.14%.

54. The injectable composition of claim 25, having a purity, such that the total impurities is less than 0.1%.

55. The injectable composition of claim 25, packaged in a container enclosing the composition, the container is fit for the sterile storage of the composition.

56. The injectable composition of claim 25, packaged in a vial, bag, or prefilled syringe.

57. The injectable composition of claim 25, packaged in a container enclosing the composition, the container is fit for the sterile storage of the composition, the container having a fill volume of 50 ml to 150 ml.
